(19) United States
(12) Patent Application Publication
(10) Pub. No.: US 2023/0347163 A1
(43) Pub. Date: Nov. 2, 2023

(54) Oral Cavity Treatment Device

(71) Applicant: Colgate-Palmolive Company, New Yorkl, NY (US)

(72) Inventors: Leighton DAVIES-SMITH, Lebanon, NJ (US); Brian Bloch, Hillsborough, NJ (US)

(73) Assignee: Colgate-Palmolive Company, New York, NY (US)

(21) Appl. No.: 18/307,401

(22) Filed: Apr. 26, 2023

Related U.S. Application Data
(60) Provisional application No. 63/337,247, filed on May 2, 2022.

(51) Int. Cl. A61N 5/06

(52) U.S. Cl. CPC A61N 5/0603; A61C 19/066; A61N 2005/0606

(57) ABSTRACT

An oral cavity treatment device which may include a mouthpiece and a power cord which extends from the mouthpiece to a plug. The power cord may be coupled to an electronic device, such as a phone or computer, to provide power to the oral cavity treatment device. The mouthpiece may include a lamp having light emitters on a flexible sheet and a lens member covering the lamp. The light emitters may be provided in staggered rows to ensure adequate tooth coverage during use. The lens may include bite protrusions positioned on opposing ends of the lens. The lens may be incredibly flexible to accommodate any mouth shape. There may be a control unit with a power button and processor located at a position along the power cord that is closer to the plug than to the mouthpiece for enhanced comfort.

### CROSS REFERENCE TO RELATED APPLICATIONS

The present application claims the benefit of priority to U.S. Provisional Pat. Application Serial No. 63/337,247, filed May 2, 2022, the entirety of which is incorporated herein by reference.

### BACKGROUND

An often-heard complaint from consumers who have invested in whitening treatments, including light emitting diode tray kits, is the perception that the process has failed to meet their whitening outcome expectations. There are many reasons that individually or in combination may account for this disappointment. It could be that the peroxide-containing formula used is ineffective, too harsh, or too painful. It could be that the light emitting tray itself is too uncomfortable, too heavy, too rigid, or causes excess salivation. All of these can contribute to a lack of procedural compliance with the treatment instructions, and therefore a potential opportunity for a failure to deliver the whitening objective. This may result in a user only revisiting the treatment sometime in the future once the memory of discomfort felt has faded. The present invention is directed to an oral cavity treatment device and related methods that address the shortcomings in the prior art.

### BRIEF SUMMARY

The present invention is directed to an oral cavity treatment device which may include a mouthpiece and a power cord which extends from the mouthpiece to a plug. The power cord may be coupled to an electronic device, such as a phone or computer, to provide power to the oral cavity treatment device. The mouthpiece may include a lamp having light emitters on a flexible sheet and a lens member covering the lamp. The light emitters may be provided in staggered rows to ensure adequate tooth coverage during use. The lens may include bite protrusions positioned on opposing ends of the lens. The lens may be incredibly flexible to accommodate any mouth shape. There may be a control unit with a power button and processor located at a position along the power cord that is closer to the plug than to the mouthpiece for enhanced comfort.

In one aspect, the invention may be an oral cavity treatment device comprising: a mouthpiece comprising: a lamp comprising a flexible sheet having a first side edge, a second side edge, and a top edge and a bottom edge each extending between the first and second side edges and a plurality of light emitters coupled to the flexible sheet, the plurality of light emitters comprising a first row of light emitters located adjacent to the top edge of the flexible sheet and arranged in a spaced apart manner in a direction between the first and second side edges of the flexible sheet, the first row of light emitters comprising a first light emitter located adjacent to the first side edge of the flexible sheet, a second light emitter located adjacent to the second side edge of the flexible sheet, and a plurality of central light emitters located between the first and second light emitters, the first and second light emitters being intersected by a first axis that extends between the first and second side edges of the flexible sheet, and wherein each of the central light emitters is located between the first axis and the top edge of the flexible sheet without being intersected by the first axis; and a lens member covering the front surface of the flexible sheet.

In another aspect, the invention may be an oral cavity treatment device comprising: a mouthpiece comprising: a lamp comprising a flexible sheet having a first side edge, a second side edge, a top edge, and a bottom edge and a plurality of light emitters coupled to the flexible sheet, the plurality of light emitters comprising a first row of light emitters adjacent to the top edge of the flexible sheet and arranged in a spaced apart manner in a direction between the first and second side edges of the flexible sheet, each of the light emitters in the first row of light emitters being staggered relative to each adjacent one of the light emitters in the first row of light emitters in a direction between the top and bottom edges of the flexible sheet; and a lens member covering the front surface of the flexible sheet.

In yet another aspect, the invention may be an oral cavity treatment device comprising: a mouthpiece comprising a lens member and a lamp having a plurality of light emitters, the lamp coupled to the lens member so that light emitted by the plurality of light emitters passes through the lens member; a power cord comprising a first end that is in contact with the lamp and a second end comprising a plug that is configured to be plugged into a power source for supplying power to the plurality of light emitters; a control unit comprising at least one actuator and a processor, wherein upon powering on the plurality of light emitters, the plurality of light emitters are configured to emit light for a predetermined duration; and wherein prior to powering on the plurality of light emitters, activation of the at least one actuator causes the processor to switch between a plurality of modes, the predetermined duration being different for each of the plurality of modes.

In still another aspect, the invention may be a method of whitening teeth comprising: selecting a tooth whitening composition from a plurality of tooth whitening compositions having different weight percentages of an active ingredient; applying the tooth whitening composition onto teeth of a user; selecting a brightness at which a plurality of light emitters of an oral cavity treatment device will emit light when activated; selecting a duration during which the plurality of light emitters of the oral cavity treatment device will emit the light when activated; positioning a mouthpiece of the oral cavity treatment device into an oral cavity of the user; and activating the oral cavity treatment device so that the plurality of light emitters emit the light onto the teeth of the user that are pre-coated with the tooth whitening composition.

In yet another aspect, the invention may be an oral cavity treatment device comprising: a mouthpiece comprising: a lamp comprising a flexible sheet having a front surface and plurality of light emitters located on the front surface of the flexible sheet; and a lens member covering the front surface of the flexible sheet and being transparent so that light emitted by the plurality of light emitters passes through the lens member, the lens member comprising a front surface having a vertical centerline, a first bite protrusion extending from the front surface of the lens member on a first side of the vertical centerline and having a first maximum width, and a second bite protrusion extending from the front surface of the lens member on a second side of the vertical centerline and having a second maximum width, an exposed portion of the front surface of the lens member located between the first and second bite protrusions being devoid of any additional bite protrusions and having a third width that is greater than each of the first and second maximum widths.

In a further aspect, the invention may be an oral cavity treatment device comprising: a mouthpiece comprising: a lamp comprising a flexible sheet having a front surface and a plurality of light emitters protruding from the front surface of the flexible sheet; and a lens member covering the front surface of the flexible sheet; and a power cord extending from the mouthpiece, the power cord comprising a plug for engaging a socket to power the plurality of light emitters, the power cord having a length; and a control unit coupled to the power cord, the control unit comprising an actuator and a processor for powering the plurality of light emitters in accordance with a treatment protocol, the control unit being located closer to the plug of the power cord than to the mouthpiece.

In a still further aspect, the invention may be an oral cavity treatment device comprising: a support member comprising a support panel having a front surface and a rear surface and a protuberance extending from the rear surface to a distal end, a passageway extending from a first opening in the front surface of the support panel to a second opening in the distal end of the protuberance, the support member comprising a first connection feature; a strain relief member disposed within the passageway, the strain relief member comprising a second connection feature that engages with the first connection feature of the support member to couple the strain relief member to the support member, the strain relief member comprising a front surface, a rear surface, a through-hole extending from the front surface to the rear surface, and at least one protrusion protruding from the front surface; a lamp comprising a flexible sheet having a front surface, a rear surface, and at least one hole extending between the front and rear surfaces and a plurality of light emitters coupled to the front surface of the flexible sheet, the rear surface of the lamp abutting the front surface of the support panel of the support member and the at least one protrusion of the strain relief member protruding through the at least one hole in the flexible sheet of the lamp; and a power cord comprising a first end having a contact element that engages the lamp and a second end comprising a plug, a first end portion of the power cord positioned within the through-hole of the strain relief member and prevented from being pulled through the through-hole of the strain relief member in a first direction due to contact between abutting surfaces of the power cord and the strain relief member.

In yet another aspect, the invention may be an oral cavity treatment device comprising: a mouthpiece comprising: a lamp comprising a flexible sheet having a front surface and a rear surface and a plurality of light emitters protruding from the front surface of the flexible sheet; a support member covering the rear surface of the flexible sheet, the support member having an exposed rear surface; and a lens member covering the front surface of the flexible sheet, the lens member having an exposed front surface; and a power cord extending from the mouthpiece, the power cord comprising a plug for engaging a socket of an electronic device to power the plurality of light emitters; wherein the mouthpiece comprises a first side portion, a second side portion, and a central portion between the first and second side portions; and wherein the mouthpiece is flexible such that each of the first and second side portions is configured to be folded relative to the central portion in a first direction so that the exposed front surface of the lens member along the first and second side portions contacts the exposed front surface of the lens member along the central portion and a second direction so that the exposed rear surface of the support member along the first and second side portions contacts the exposed rear surface of the support along the central portion.

In yet another aspect, the invention may be an oral cavity treatment device comprising: a mouthpiece comprising: a lamp comprising a flexible sheet having a front surface and plurality of light emitters located on the front surface of the flexible sheet; and a lens member covering the front surface of the flexible sheet and being transparent so that light emitted by the plurality of light emitters passes through the lens member, the lens member comprising: a front surface having a vertical centerline, a first bite protrusion extending from the front surface of the lens member on a first side of the vertical centerline, the first bite protrusion having a first side edge opposite a second side edge, and a second bite protrusion extending from the front surface of the lens member on a second side of the vertical centerline, the second bite protrusion having a first side edge opposite a second side edge, an exposed portion of the front surface of the lens member located between the first and second bite protrusions being devoid of any additional bite protrusions; wherein a side edge axis intersects the second side edge of the first bite protrusion and the second side edge of the second bite protrusion; and wherein the second side edge of the first bite protrusion and the second side edge of the second bite protrusion are separated by a first distance when a first external force is applied to the second side edge of the first bite protrusion and the second side edge of the second bite protrusion along the side edge axis; and wherein the second side edge of the first bite protrusion and the second side edge of the second bite protrusion are separated by a second distance when a second external force is applied to the second side edge of the first bite protrusion and the second side edge of the second bite protrusion along the side edge axis; and wherein the first external force is zero; and wherein the second distance is about 5 mm less than the first distance when the second external force is less than about 1.5 N.

In another aspect, the invention may be an oral cavity treatment device comprising: an arched mouthpiece comprising a lamp, wherein a first distance exists between first and second side edges of the arched mouthpiece when the arched mouthpiece is in a normal state; and the arched mouthpiece configured to flex so that the first distance decreases by at least 5 mm upon an external compression force being applied to the first and second side edges that is less than or equal to about 1.5 N.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of an oral cavity treatment device in accordance with an embodiment of the present invention;

FIG. 2 is a perspective view of the oral cavity treatment device of FIG. 1 with a cover thereof removed;

FIG. 3 is an exploded perspective view of the oral cavity treatment device of FIG. 1;

FIG. 4 is a cross-sectional view taken along line IV-IV of FIG. 1;

FIG. 5 is a close-up view of area V of FIG. 4;

FIG. 6 is a cross-sectional view taken along line VI-VI of FIG. 1;

FIG. 7 is a perspective view of a lamp of the oral cavity treatment device of FIG. 1;

FIG. 8 is a front view of the lamp of FIG. 7;

FIG. 9 is a perspective view of a lens member of the oral cavity treatment device of FIG. 1;

FIG. 10 is a rear perspective view of the lens member of FIG. 9;

FIG. 11 is a view illustrating a power cord of the oral cavity treatment device of FIG. 1 plugged into an electronic device;

FIG. 12 is the view of FIG. 11 with a mouthpiece of the oral cavity treatment device located within a user’s oral cavity;

FIGS. 13A and 13B illustrate a mode adjustment feature of the oral cavity treatment device of FIG. 1; and

FIG. 14 shows the oral cavity treatment device in a testing arrangement according to the present invention.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

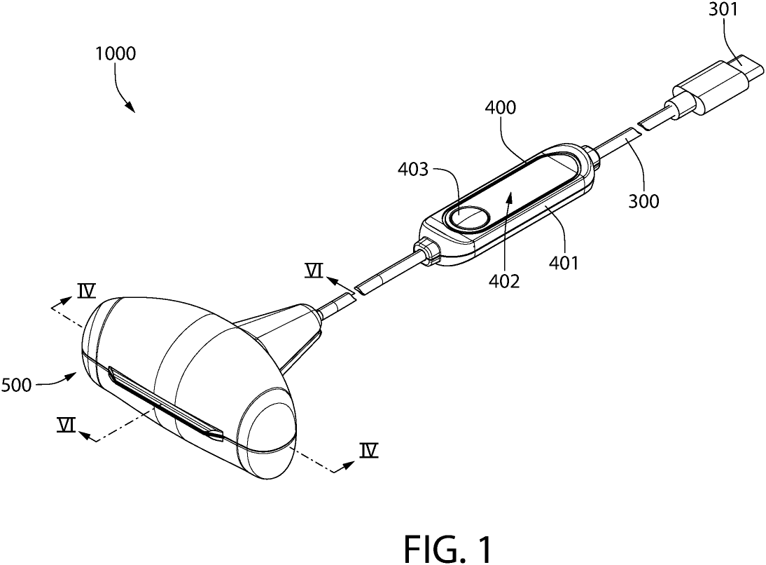

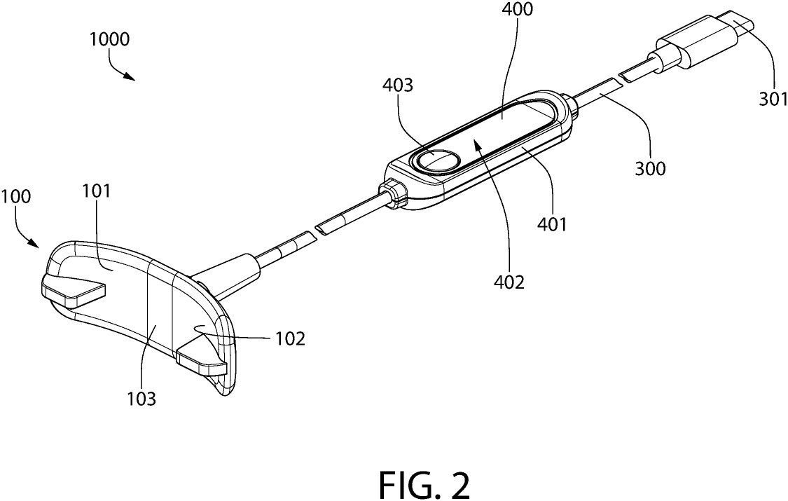

Referring first to FIGS. 1 and 2, an oral cavity treatment device 1000 will be described in accordance with an embodiment of the present invention. The oral cavity treatment device 1000 may be configured to be used within a user’s oral cavity to emit electromagnetic radiation or light onto the user’s teeth and/or gums to perform or enhance a treatment thereon. In one embodiment, the oral cavity treatment device 1000 may be used in conjunction with a tooth whitening formulation that may be applied onto the teeth prior to enhancement with the oral cavity treatment device 1000. One particular tooth whitening composition which may be used in conjunction with the oral cavity treatment device 1000 is described in U.S. Pat. No. 11,040,002. However, the oral cavity treatment device 1000 may be used alone (i.e., without first coating the teeth with a tooth whitening composition) in some embodiments, and in other embodiments the oral cavity treatment device 1000 may be used after the user coats the teeth with any tooth whitening composition, such as for example without limitation those containing hydrogen peroxide or carbamide peroxide. The tooth whitening composition which may be used in conjunction with the oral cavity treatment device 1000 to whiten the teeth is not limited to the specific formulation described in U.S. Pat. No. 11,040,002 and any whitening formulation now known or later discovered which may benefit from light treatment after application to the teeth may be used. Moreover, other formulations, such as anti-sensitivity formulations, fresh breath formulations, or the like which may similarly benefit from a light treatment after application of the formulation to the teeth may be used with the oral cavity treatment device 1000. One additional example composition is one which includes monoperoxisulfate, which is an oxidizing agent that has a positive reaction with the light being emitted from the device. Furthermore, Vitamin C compounds acting as a reducing agent for stain removal may also be used. Finally, the oral care treatment device 1000 may emit light at approximately 410 nm which may have antibacterial benefits.

The oral cavity treatment device 1000 generally comprises a mouthpiece 100, a power cord 300, and a control unit 400. The mouthpiece 100 is the part of the oral cavity treatment device 1000 that is placed within the user’s oral cavity during a treatment procedure. The power cord 300 extends from the mouthpiece 100 and terminates in a plug 301 that may be plugged into a socket of a device which contains a power supply to supply power to the mouthpiece 100, as described herein. The oral cavity treatment device 1000 does not have its own power source, but is instead plugged into a socket of another device (which may be an electronic device such as a smart phone, a laptop, a computer, a television, a power bank, an electronic device docking station, or the like or may be a power outlet). Thus, the other device supplies power to the mouthpiece 100 and any electronic components thereof (such as light emitters as described herein) when the plug 301 is engaged with a socket of the electronic device. In the exemplified embodiment, the plug 301 is a USB-C plug. However, the invention is not to be so limited and the plug 301 may take on any structural form desired to enable the power cord 300 to be electronically coupled to an electronic device. Thus, the plug may be a USB Type A, USB Type B, USB 2.0, USB 3.0, USB Mini, USB Micro, USB Micro B, USB Type-C, a standard type plug for engagement with a power outlet of any type depending on geographic location, or the like.

The control unit 400 is located along the power cord 300. As described below, the control unit 400 may be located along the power cord 300 at a position that is closer to the plug 301 than to the mouthpiece 100. This may enhance the comfort to a user during use by not having the weight of the control unit 400 located too close to the mouthpiece 100. As such, the user will not feel the weight of the control unit 400 during use, making it easier to maintain the mouthpiece 100 in the mouth for longer periods of time. The entire oral cavity treatment device 1000 may be of a very low weight. The weight of the oral cavity treatment device 1000 may be between 10 g and 50 g, more specifically between 15 g and 30 g, and more specifically between 15 g and 25 g. This enhances the comfort of the oral cavity treatment device 1000 when in use because the user does not carry much weight in his or her mouth during use.

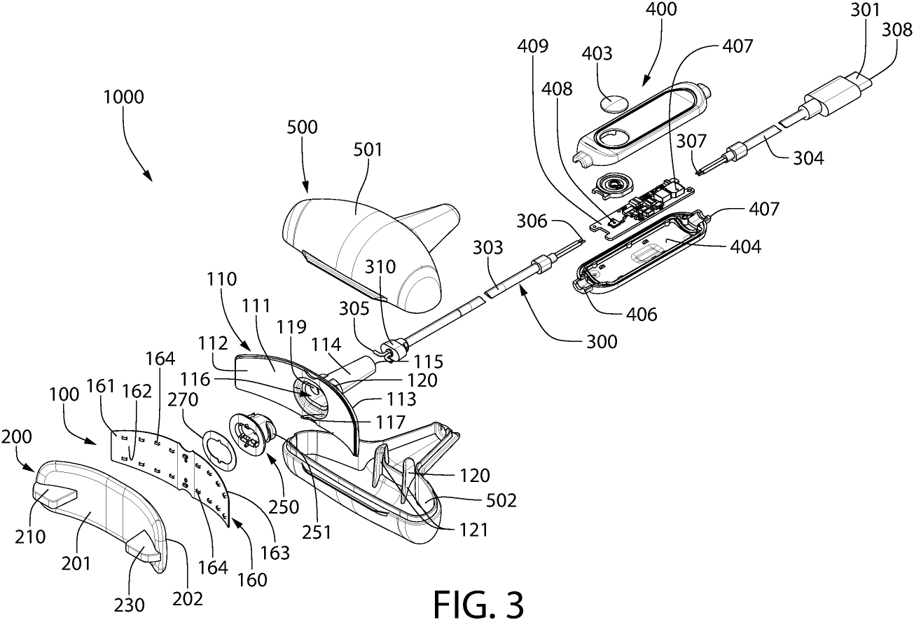

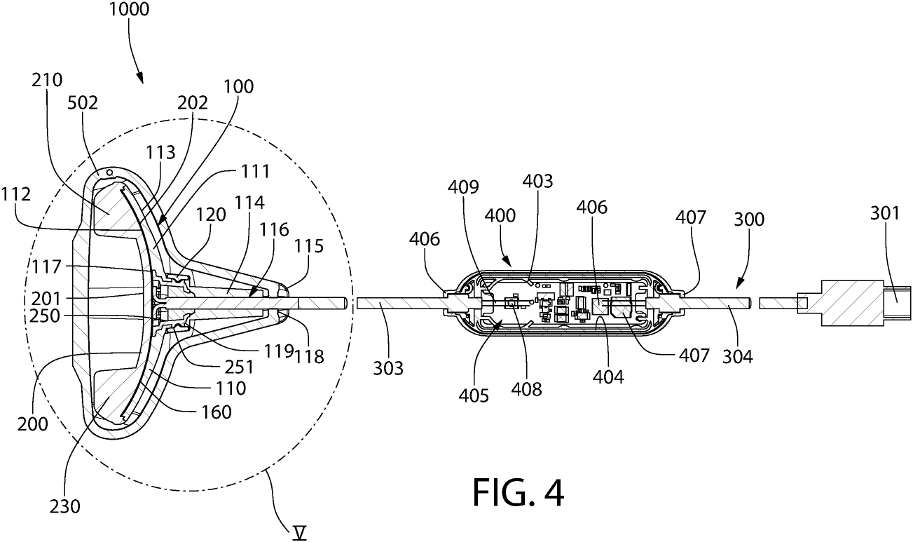

The control unit 400 comprises a housing 401 having an outer surface 402 and an inner surface 404 which defines a cavity 405 (the inner surface 404 and the cavity 405 are shown in FIGS. 3 and 4). There is at least one actuator 403 located along the outer surface 402 of the housing 401. In the exemplified embodiment the at least one actuator 403 is a depressible button that may be used to power the oral cavity treatment device 1000 on and off and may also be used to modify a mode of operation of the oral cavity treatment device 1000, as described in greater detail below. The invention is not to be so limited in all embodiments and the at least one actuator 403 may be a slide switch, a conductive switch, a clickable switch, or other implementations in other embodiments. Moreover, while a single actuator 403 is depicted in the drawings, more than one actuator may be used in other embodiments with each actuator performing a different function (on/off and mode selection, for example). The housing 401 may include a display on its outer surface 402 for visually displaying information to a user. The display may display images, text, symbols, light indicators, or the like in various different embodiments.

While the at least one actuator 403 may be used to power the oral cavity treatment device 1000 on and off, it may not function unless the plug 301 of the power cord 300 is electrically coupled to an electronic device, such as any of the types of devices described above. This is because, as noted herein, the oral cavity treatment device 1000 does not contain its own power source. This is to keep costs for the oral cavity treatment device 1000 down, negate the need for replacement of batteries, and keep the oral cavity treatment device 1000 at a very low weight. Thus, activating the actuator 403 without the plug 301 being electrically coupled to an electronic device will not result in any powering on of the device. However, when the plug 301 is electrically coupled to an electronic device, activation of the actuator 403 will either change a mode of operation and/or power the device on/off, as described further herein below.

In FIG. 1, a cover 500 is illustrated positioned over the mouthpiece 100. The cover 500 encloses the mouthpiece 100 to protect against damage and to prevent bacteria or the like from coating the mouthpiece 100 during periods of non-use. Thus, when not in use, the mouthpiece 100 may be enclosed within the cover 500. The cover 500 may be detached from the mouthpiece 100 to allow the mouthpiece 100 to be inserted into the user’s mouth or oral cavity to perform a treatment. The cover 500 may be a single part that opens and closes at a hinge, such as a living hinge. Alternatively, the cover 500 may be formed from two separate cover portions that may be snapped together and detached from one another by the user. While a particular structural shape is shown in the drawings, the cover 500 may have other shapes in other embodiments so long as it is suited for its function as described herein. The cover 500 may also be omitted entirely in some embodiments such that the oral cavity treatment device 1000 may not include the cover 500.

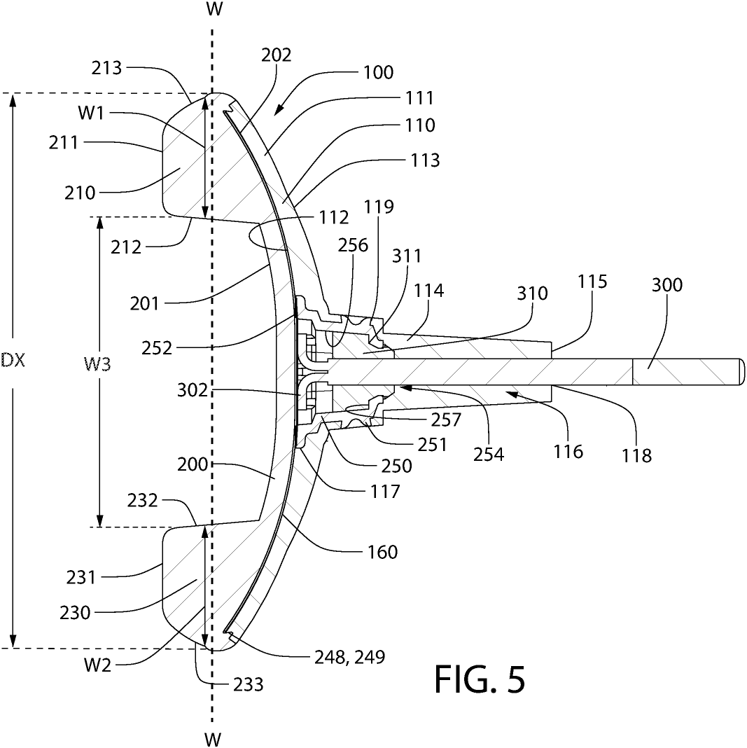

Turning to FIGS. 3-6, the oral cavity treatment device 1000 will be described in further detail. The mouthpiece 100 comprises a support member 110, a lamp 160, and a lens member 200 that are securely and generally non-detachably coupled together so that the mouthpiece 100 is a unitary and integral part of the oral cavity treatment device 1000. That is, while the support member 100, the lamp 160, and the lens member 200 are separate components, due to the assembly of the mouthpiece 100 the various components cannot be separated from one another, absent tearing or otherwise destroying one or more of the various components. The support member 110 and the lens member 200 may be bonded together during the manufacturing process, such that one of the support member 110 and the lens member 200 may be injection molded onto the other to create a bond between the materials. Alternatively, the lens member 200 and the support member 110 may be adhesively bonded to one another by applying an adhesive material onto the interfacing surfaces (or portions thereof) of the lens member 200 and the support member 110. The support member 110 and the lens member 200 may also have engaging edges and surfaces that interlock the two components together once manufactured to create a mechanical interlock between the two parts, as best shown in FIG. 5 and described further below.

The support member 110 comprises a support panel 111 having a front surface 112 and a rear surface 113. The support member 110 further comprises a protuberance 114 that extends from the rear surface 113 of the support panel 111 to a distal end 115. The front surface 112 of the support panel 111 may be slightly concave. In other embodiments, the front surface 112 of the support panel 111 may be flat/planar rather than concave, and the support panel 111 may be curved during use due to its flexibility. The support member 110 may be formed from an elastomeric material, such as a thermoplastic elastomer, having a low Shore A hardness which provides the support member 110 with a high degree of flexibility. The support member 110 may be formed from a thermoplastic elastomer having a Shore A hardness between 30 and 40, and more specifically of approximately 35. This is similar to the Shore A hardness of a rubber band, which means that the support member 110 is quite flexible. The thickness of the support member may be between 0.5-2 mm, which adds to the flexibility. One example of a material which may be used to form the support member 110 is MEDALIST® MD-10118 NAT or MEDALIST® MD-10108 NAT sold by Teknor Apex.

As mentioned above, the protuberance 114 protrudes from the rear surface 113 of the support panel 111, which is convex in the exemplified embodiment but could be flat in other embodiments. The protuberance 114 may have a tubular shape and may be conical such that its diameter may taper extending from the support panel 111 to the distal end 115. The specific shape of the protuberance 114 is not to be limiting of the invention in all embodiments and the protuberance 114 may take on other shapes, such as having a polygonal cross-sectional shape rather than a circular cross-sectional shape as in the exemplified embodiment.

The support member 110 may comprise a passageway 116 extending from a first opening 117 in the front surface 112 of the support panel 111 to a second opening 118 in the distal end 115 of the protuberance 114. The passageway 116 may be configured to receive and hold a portion of the power cord 300 while maintaining an end of the power cord 300 in contact with the lamp 160 so that power can be supplied to light emitters of the lamp 160, as described further herein.

The support member 110 comprises a first connection feature 119 to facilitate coupling of a strain relief member 250 thereto, as described further below. In the exemplified embodiment, the first connection feature 119 comprises one or more apertures formed through the protuberance 114 of the support member 110 which may receive and/or be engaged by protrusions of the strain relief member 250 to couple the strain relief member 250 thereto. The cover 500 may comprise a snap-fit connector 120 that snap-fit connects to the strain relief member 250 to facilitate the coupling between the cover 500 and the oral cavity treatment device 1000 when the cover 500 is coupled thereto. The snap-fit connector 120 of the cover 500 may comprise first and second snap-fit arms 121 that are spaced apart by a gap. When the cover 500 encloses the mouthpiece 100, the portion of the mouthpiece 100 where the strain relief member 250 is exposed through the apertures of the first connection feature 119 of the support member 110 nests within the gap between the snap-fit arms 121. The snap-fit arms 121 may be protuberances or recesses that engage recesses or protuberances of the portion of the strain relief member 250 which are exposed through the apertures of the first connection feature 119 of the support member 110, as best shown in FIG. 4.

The lamp 160 generally comprises a flexible sheet 161 and a plurality of light emitters 164. The flexible sheet 161 has a front surface 162 and a rear surface 163. The front surface 162 may be slightly concave and the rear surface 163 may be slightly convex. Alternatively, the front and rear surfaces may be flat/planar such that they are concave and convex only when flexed during use by the user to correspond to the dental arch. The flexible sheet 161 may be a flexible printed circuit board (PCB). The flexible sheet 161 is able to bend, flex, or the like in response to forces being applied thereto. Thus, when the mouthpiece 100 is placed into the user’s mouth, the user can flex/bend the entire mouthpiece 100 to match the curve of the user’s dental arch.

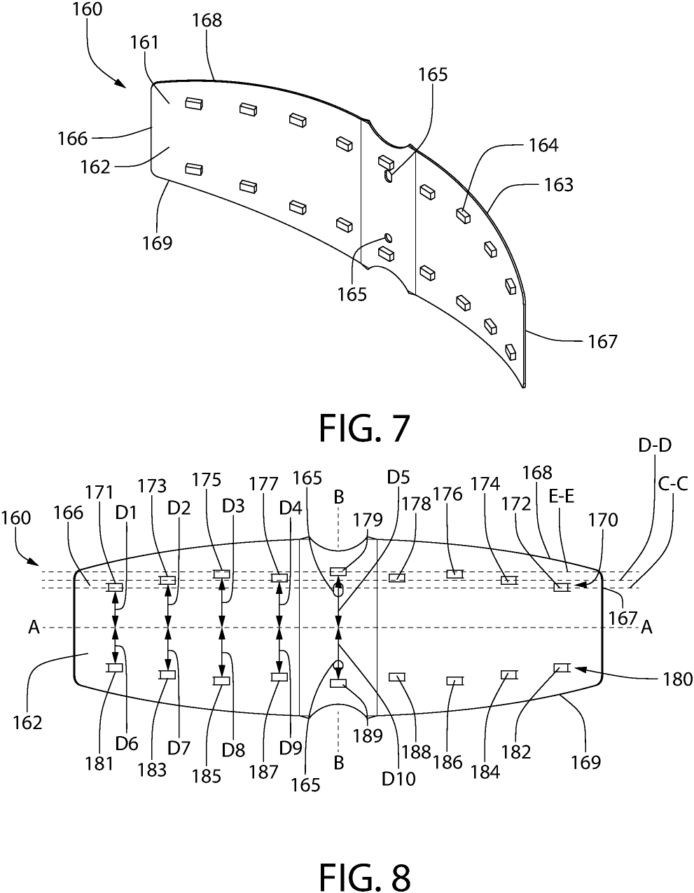

The plurality of light emitters 164 are coupled to the flexible sheet 161 and are located along the front surface 162 of the flexible sheet 161. The plurality of light emitters 164 may be light emitting diodes (LEDs). However, the invention is not to be so limited in all embodiments and the plurality of light emitters 164 may be other types of light sources in other embodiments. In one embodiment, the light emitters 164 may be formed as printed light emitting diodes, such as those manufactured by NthDegree. The plurality of light emitters 164 may comprise a plurality of discrete light emitters that are positioned in a spaced apart manner along the front surface 162 of the flexible sheet 161. The plurality of light emitters 164 may protrude from the front surface 162 of the flexible sheet 161 in some embodiments. Although not illustrated, an electrical contact may exist on the rear surface 163 of the flexible sheet 161 so that power from a power source of an external device may be transmitted through the power cable 300 to the plurality of light emitters 164. That is, an exposed electrical conductor 302 of the power cord 300 may be in direct contact with the electrical contact on the rear surface 163 of the flexible sheet 161. The electrical contact on the rear surface 163 of the flexible sheet 161 may in turn be electrically coupled to each of the light emitters 164 (directly to some and indirectly to others, or directly to all) to transmit power to the light emitters 164 so that they may be powered to emit light during a treatment protocol. Additional details related to the lamp 160 will be provided below with reference to FIGS. 7 and 8.

The lens member 200 is configured to cover the lamp 160 so that the front surface 162 of the flexible sheet 161 as well as the light emitters 164 are not exposed to the elements or to the user’s oral cavity during use. Rather, the lamp 160 is sandwiched between the lens member 200 and the support member 110 as discussed herein. Thus, the lens member 200 and the support member 110 may be bonded together to create a hermetically sealed cavity within which the lamp 160 is located so that the user’s saliva and other contaminants may not enter into the cavity and damage the lamp 160 and other electronic components of the oral cavity treatment device 1000. The lens member 200 is preferably formed from a thermoplastic elastomer, and may be formed from the same thermoplastic elastomer as the support member 110 (the same material construction, although it may not be the same color). Thus, the lens member 200 may have a Shore A hardness between 30 and 40, and more specifically approximately 35.

Thus, the lens member 200, the lamp 160, and the support panel 111 of the support member 110 are very flexible, and the entire mouthpiece 100 may be readily flexed in order to fit the contours/curvature of a particular user’s dental arch. This flexibility of the mouthpiece 100 allows the mouthpiece 100 to be comfortably used by virtually any person regardless of the particular curvature of that person’s dental arch. To explain the flexibility of the mouthpiece 100, with reference to FIG. 2, imagine that the mouthpiece 100 comprises a first side portion 101, a second side portion 102, and a central portion 103 located between the first and second side portions. The first and second side portions 101, 102 may be bent or folded relative to the central portion 103 to such a degree that the front surface 201 of the lens member 200 along the first and second side portions 101, 102 may contact the front surface 201 of the lens member 200 along the central portion 103. Bite protuberances 210, 230 of the lens member 200, described below, may get in the way of this bending/folding, but the bite protuberances 210, 230 may also be bent out of the way to allow for this contact. The first and second side portions 101, 102 may also be bent/flexed rearwardly relative to the central portion 103 so that the rear surface 113 of the support member 110 along the first and second side portions 101, 102 may contact the rear surface 113 of the support member 110 along the central portion 103. Thus, the first and second side portions 101, 102 may be folded approximately 180° (plus or minus 10°) relative to the central portion 103 in either direction. This is an incredible amount of flexibility which allows the mouthpiece 100 to readily fit the contours of virtually any human oral cavity dental arch.

The lens member 200 may be formed from a clarified thermoplastic elastomer. Stated another way, the lens member 200 may be transparent. As such, any light emitted by the light emitters 164 will readily pass through the lens member 200 and onto the user’s teeth. The lens member 200 is fully transparent to the light being emitted by the light emitters 164 (which might be light having a wavelength between 390 nm and 430 nm, more specifically between 400 nm and 420 nm, and still more specifically approximately 410 nm, approximately in this context meaning plus or minus 5 nm). It is worth noting that the support member 110 may be formed from an opaque color, such as white, red, green, blue, black, or the like, or the support member 110 may also be transparent if so desired. However, there may not be a requirement for the support member 110 to be transparent because the light emitted by the light emitters 164 is not intended to transmit through the support member 110. In some embodiments, the support member 110 may be transparent so that the light emits therethrough, to provide for an aesthetic effect. The lens member 200 may include facets on its front and/or rear faces to help with the dispersion of the light emitted by the light emitters 164.

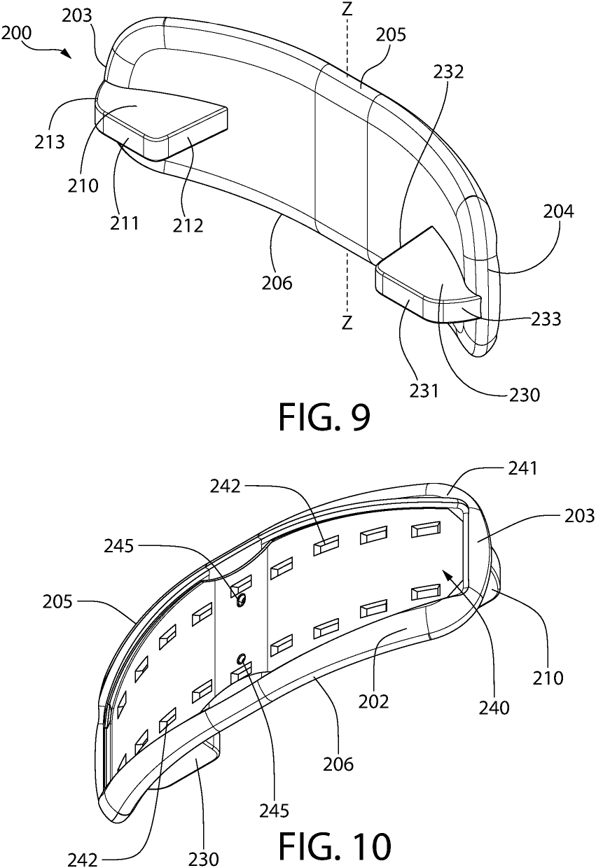

The lens member 200 comprises a front surface 201 and a rear surface 202. The front surface 201 may be concave and the rear surface 202 may be convex. Of course, as with the other components of the mouthpiece 100, the front and rear surfaces 201, 202 may be flat/planar in other embodiments and the curvature may only be achieved when the device is placed in the user’s oral cavity. As shown in FIGS. 3 and 4 and mentioned above, the lens member 200 may comprise a first bite protrusion 210 and a second bite protrusion 230 extending from the front surface 201 of the lens member 200 in a spaced apart manner. Additional details about the first and second bite protrusions 210, 230 will be provided below with reference to FIGS. 9 and 10.

The oral cavity treatment device 1000 further comprises the strain relief member 250 (mentioned above) and a double-sided adhesive panel 270. The strain relief member 250 is positioned within the passageway 116 of the support member 110 to facilitate locking the power cord 300 into position and preventing detachment of the power cord 300 from the mouthpiece 100. The strain relief member 250 comprises a second connection feature 251 that engages the first connection feature 119 of the support member 110 to lock the strain relief member 250 to the support member 110 within the passageway 116. In the exemplified embodiment, the second connection feature 251 is a protrusion that extends into/nests within the aperture of the first connection feature 119 to lock the strain relief member 250 into place. Thus, the second connection feature 251 of the strain relief member 250 may be exposed through the aperture of the first connection feature 119 of the support member 110. The strain relief member 250 may be formed from a hard plastic material (ABS, polypropylene, or similar hard plastics), metal, or other rigid materials.

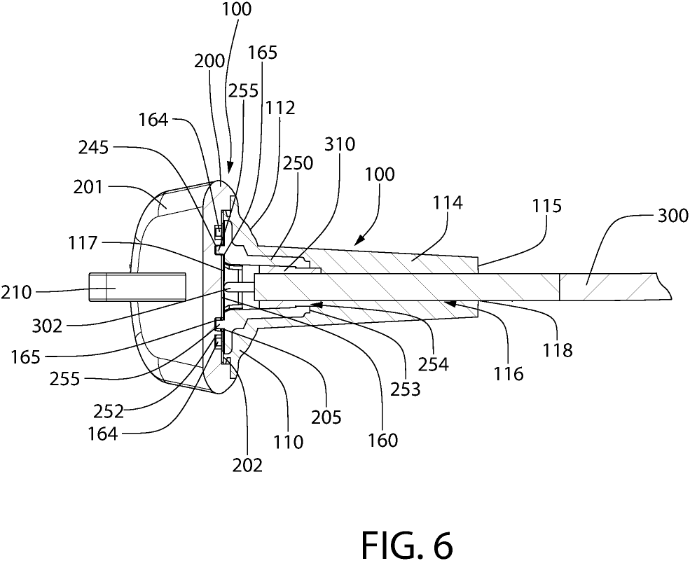

The strain relief member 250 may comprise a front surface 252, a rear surface 253, and a through-hole 254 extending from the front surface 252 to the rear surface 253. The strain relief member 250 comprises at least one protrusion 255 protruding from the front surface 252. In the exemplified embodiment, there are two of the protrusions 255, but there could be just one or more than two in other embodiments. As best seen in FIG. 6 (and also FIGS. 7 and 8), the lamp 160 comprises at least one hole 165. In the exemplified embodiment, the lamp 160 comprises two of the holes 165. In the exemplified embodiment, the protrusions 255 of the strain relief member 250 extend into and through the holes 165 in the lamp 160. Thus, because the strain relief member 250 is secured in place within the passageway 116 and is fixed/non-movable relative to the support member 110, the engagement of the protrusions 255 of the strain relief member 250 with the holes 165 in the lamp 160 serves to hold the lamp 160 in place and prevent the lamp 160 from moving within the space between the support member 110 and the lens member 200.

In the exemplified embodiment, the lens member 200 also comprises at least one blind hole 245 formed into the rear surface 202 which is aligned with the protrusions 255 of the strain relief member 250 and the holes 165 in the lamp 160 when the mouthpiece 100 is assembled. The protrusions 255 of the strain relief member 250 may extend through the holes 165 in the lamp 160 and distal portions of the protrusions 255 of the strain relief member 250 may nest within the blind holes 245 in the lens member 200. The blind holes 245 in the rear surface 202 of the lens member 200 are further illustrated in FIG. 10.

The strain relief member 250 nests within the passageway 116 of the support member 110 and the front surface 252 of the strain relief member 250 is essentially flush (plus or minus 0.5 mm) with the front surface 112 of the support panel 111 of the support member 110. The double sided adhesive panel 270 may be positioned between the front surface 252 of the strain relief member 250 and the rear surface 163 of the flexible sheet 161 of the lamp 160 to further secure the attachment of the various parts. However, the double-sided adhesive panel 270 may be omitted in some embodiments.

As best shown in FIG. 5, the strain relief member 250 comprises an inner surface 256 which defines the through-hole 254. The inner surface 256 of the strain relief member 250 comprises a ledge 257 that faces upwardly towards the front surface 252. The power cord 300 comprises an extension collar 310 that is positioned around the main body of the power cord 300 adjacent to the electrical conductor 302. The extension collar 310 may be bonded to the main body of the power cord 300 so that the main body of the power cord cannot be slid or otherwise moved relative to the extension collar 310. The extension collar 310 comprises a shoulder 311 that faces away from the electrical conductor 302 and towards the plug 301.

When the oral cavity treatment device 1000 is assembled, the extension collar 310 of the power cord 300 nests within the through-hole 254 of the strain relief member 250. Furthermore, the shoulder 311 of the extension collar 310 abuts against the ledge 257 of the inner surface 256 of the strain relief member 250. As such, the power cord 250 cannot be pulled through the through-hole 254 relative to the strain relief member 250 in the direction of the plug 301 due to the abutment of the shoulder 311 with the ledge 257. The only way to remove the power cord 250 would be if the strain relief member 250 were to disengage from the support member 210. However, as seen in FIG. 5, the strain relief member 250 and the support panel 211 of the support member 210 have engaging features and surfaces (including the first and second connection features 119, 251, and potentially other engaging surfaces) that prevent the strain relief member 250 from being separated from the support panel 211 of the support member 210. Thus, this engagement/abutment/coordination between the surfaces serves to maintain the power cord 300 in position with the electrical conductor 302 in contact with an electrical contact on the rear surface of the lamp 160. It is possible that the power cord 300 may be able to move/slide relative to the support member 110 in the direction away from the plug 301. However, the lens member 200 prevents significant movement of the power cord 300 in this direction due to the bonding or other attachment of the lens member 200 to the support member 110 as described herein.

The oral cavity treatment device 1000 may be assembled in the following order. First, the strain relief member 250 is positioned within the passageway 116 of the support member 210. This may be achieved by sliding the strain relief member 250 into the passageway 116 from the opening 117 in the front surface 112 of the support panel 111, or by injection molding the support member 110 directly onto and around the strain relief member 250. Next, the power cord 300 (or a portion thereof) is slid through the through-hole 254 of the strain relief member 250 in a direction moving from the front surface 112 of the support panel 111 to the rear surface 113 of the support panel 111 until the shoulder 311 abuts against the ledge 257. Next, the adhesive panel 270 may be adhered to the front surface 252 of the strain relief member 250 (although this step may be omitted). Next, the lamp 160 may be positioned with its rear surface 163 abutting the front surface 112 of the support panel 111. The adhesive panel 270 may serve to hold the lamp 160 in this position. The lamp 160 may be positioned so that the electrical conductor 302 of the power cord 300 is in contact with an electrical contact on the rear surface 163 of the lamp 160. Finally, the lens member 200 may be injection molded onto the lamp 160 and support panel 111 to form the mouthpiece 100. At this point, the lamp 160 is sandwiched between the support panel 111 and the lens member 200 and the power cord 300 is locked in place with a portion of the power cord 300 located within the passageway 116 and another portion of the power cord 300 protruding from the distal end 115 of the protuberance 114 of the support member 110. Alternatively, the lens member 200 may be separately formed and then bonded/attached to the support member 110 using adhesives or the like.

As best shown in FIG. 3, the power cord 300 may comprise a first cord portion 303 and a second cord portion 304, with the control unit 400 being located between the first and second cord portions 303, 304. The first cord portion 303 extends from a first end 305 to a second end 306 and the second cord portion 303 extends from a first end 307 to a second end 308. The first end 305 of the first cord portion 302 comprises the electrical conductor 302. The second end 306 of the first cord portion 302 is coupled to a proximal end 406 of the control unit 402. The second end 308 of the second cord portion 303 comprises the plug 301. The first end 307 of the second cord portion 303 is coupled to a distal end 407 of the control unit 402.

In the exemplified embodiment, the first cord portion 303 has a first length measured from the first end 305 to the second end 306 and the second cord portion 304 has a second length measured from the first end 307 to the second end 308. The first length may be greater than the second length. In some embodiments, the first length may be at least two times the second length. In some embodiments, the power cord 300 may have a length, and the control unit 400 may be located along a distal-one third of the length of the power cord 300. The power cord 300 may have a length between 30 inches and 40 inches, and more specifically between 32 inches and 36 inches. In such embodiment, the control unit 400 may be located no more than 15 inches from the plug 301. In other embodiments, the control unit 400 may be located no more than 12 inches from the plug 301. In some embodiments, the control unit 400 may be located between 8 inches and 12 inches from the plug 301. Thus, the control unit 400 is located closer the second end 308 of the second cord portion 304 than to the first end 305 of the first cord portion 303. Or, as noted above, the control unit 400 is located closer to the plug 301 than to the mouthpiece 100. This ensures that the user is not supporting the weight of the control unit 400 when the mouthpiece 100 is in the user’s oral cavity, which may occur if the control unit 400 were located closer to the mouthpiece 100 than to the plug 301.

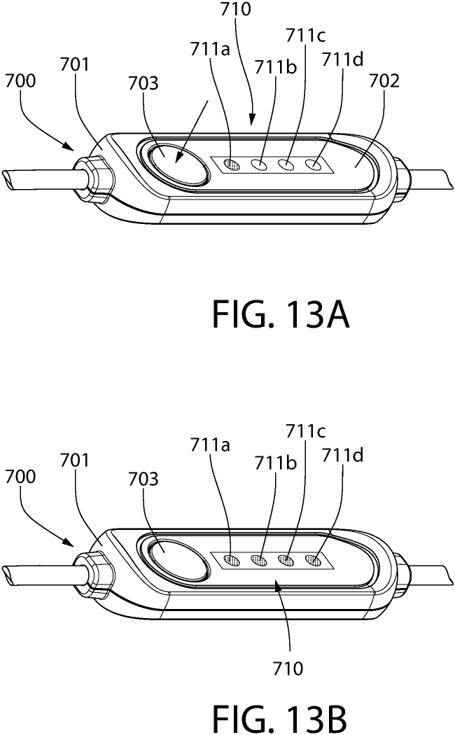

The control unit 400 comprises the actuator 403 and other circuitry and/or electronic components operably coupled to the actuator 403 and located within the cavity 405 of the housing 401. The actuator 403 may comprise or be operably coupled to a switch 408 that is located on a printed circuit board 409. Furthermore, the control unit 400 may comprise a processor 406 and a timer 407 that are operably coupled together and to the switch 408. Additional electronic components which may be necessary for the operation and function of the oral cavity treatment device 1000 may also be included on the printed circuit board 409 housed within the cavity 405 of the housing 401. The processor 406 and the timer 407 may be located on the printed circuit board 409. Thus, when the power cord 300 is plugged in and the user activates the actuator 403, the processor may cause the light emitters 164 of the lamp 160 to emit for a predetermined period of time, as determined by the timer 407. The predetermined period of time may be preset in some embodiments and it may be adjustable in other embodiments. That is, a user may be able to adjust or otherwise change the ON time for the light emitters 164 for a particular tooth whitening session. This will be discussed in more detail below with reference to FIGS. 13A and 13B.

Referring to FIGS. 7 and 8, the lamp 160 will be further described. As noted, the lamp 160 comprises the flexible sheet 161 and the plurality of light emitters 164. The flexible sheet 161 comprises the front surface 162 and the rear surface 163, and the plurality of light emitters 164 are located on the front surface 162. The plurality of light emitters 164 may protrude from the front surface 162. The flexible sheet 161 also comprises the at least one hole 165 which extends from the front surface 162 to the rear surface 163. There are two of the holes 165 in the exemplified embodiment for receiving the two protrusions 255 of the strain relief member 250 as discussed above.

The flexible sheet 161 comprises a first side edge 166, a second side edge 167, a top edge 168, and a bottom edge 169. The top and bottom edges 168, 169 each extend between the first and second side edges 166, 167. The flexible sheet 161 comprises a longitudinal axis A-A which extends between the first and second side edges 166, 167 at a position that is equidistant to the top and bottom edges 168, 169. The flexible sheet 161 further comprises a vertical centerline B-B extending between the top and bottom edges 168, 169 of the flexible sheet 161 in a location that is equidistant to the first and second side edges 166, 167 of the vertical sheet 161. The plurality of light emitters 164 comprises a first row of light emitters 170 located adjacent to the top edge 168 of the flexible sheet 161 between the longitudinal axis A-A and the top edge 168 of the flexible sheet 161 and a second row of light emitters 180 located adjacent to the bottom edge 169 of the flexible sheet 161 between the longitudinal axis A-A and the bottom edge 169 of the flexible sheet 161.

The light emitters 164 of the first row 170 are staggered relative to one another in a direction between the top and bottom edges 168, 169 of the flexible sheet 161. That is, each of the light emitters 164 of the first row 170 is staggered in the direction of the top/bottom edges 168, 169 of the flexible sheet 161 relative to each of the light emitters 164 of the first row 170 that are adjacent to it. Thus, no two adjacent light emitters 164 of the first row 170 are perfectly aligned on an axis that is parallel to the longitudinal axis A-A. Moreover, the distance between the light emitters 164 of the first row 170 and the longitudinal axis A-A does not continually increase moving from the first and second sides 166, 167 inwardly towards the vertical axis B-B. Rather, the distance between some of the adjacent light emitters 164 of the first row 170 and the longitudinal axis A-A increases moving from the first side edge 166 and the second side edge 167 towards the vertical axis B-B, and the distance between some of the adjacent light emitters 164 of the first row 170 and the longitudinal axis A-A decreases moving from the first side edge 166 and the second side edge 167 towards the vertical axis B-B.

The light emitters 164 of the second row 180 are staggered relative to one another in a direction between the top and bottom edges 168, 169 of the flexible sheet 161. That is, each of the light emitters 164 of the second row 180 is staggered in the direction of the top/bottom edges 168, 169 of the flexible sheet 161 relative to each of the light emitters 164 of the second row 180 that are adjacent to it. Thus, no two adjacent light emitters 164 of the second row 180 are perfectly aligned on an axis with is parallel to the longitudinal axis A-A. Moreover, the distance between the light emitters 164 of the second row 180 and the longitudinal axis A-A does not continually increase moving from the first and second sides 166, 167 inwardly towards the vertical axis B-B. Rather, the distance between some of the adjacent light emitters 164 of the second row 180 and the longitudinal axis A-A increases moving from the first side edge 166 and the second side edge 167 towards the vertical axis B-B, and the distance between some of the adjacent light emitters 164 of the second row 180 and the longitudinal axis A-A decreases moving from the first side edge 166 and the second side edge 167 towards the vertical axis B-B.

The light emitters 164 of the first row 170 comprises a first light emitter 171 positioned closest to the first side edge 166 of the flexible sheet 161, a second light emitter 172 positioned closest to the second side edge 167 of the flexible sheet 161, and a plurality of central light emitters located between the first and second light emitters 171, 172. The first and second light emitters 171, 172 of the first row 170 are aligned on and intersected by a first axis C-C that is parallel to the longitudinal axis A-A. Each of the central light emitters that is located between the first and second light emitters 171, 172 is located between the first axis C-C and the top end 168 of the flexible sheet 161 without being intersected by the third axis C-C.

The central light emitters of the first row 170 may comprise, moving in a direction from the first side edge 166 inwardly towards the vertical axis B-B, a third light emitter 173 adjacent to the first light emitter 171, a fifth light emitter 175 adjacent to the third light emitter 173, and a seventh light emitter 177 adjacent to the fifth light emitter 175. Furthermore, the central light emitters of the first row 170 comprises, moving in a direction from the second side edge 167 inwardly towards the vertical axis B-B, a fourth light emitter 174 adjacent to the second light emitter 172, a sixth light emitter 176 adjacent to the fourth light emitter 174, and an eighth light emitter 178 adjacent to the sixth light emitter 176. Finally, in the exemplified embodiment there is a ninth light emitter 179 between the seventh and eighth light emitters 177, 178.

A second axis D-D that is parallel to the longitudinal axis A-A may intersect the third and fourth light emitters 173, 174 and the seventh and eighth light emitters 177, 178, with the fifth, sixth, and ninth light emitters 175, 176, 179 being located between the second axis D-D and the top edge 168 of the flexible sheet 161. While the second axis D-D may intersect the third, fourth, seventh, and eighth light emitters 173, 174, 177, 178, those light emitters may not all be aligned. Rather, the third and fourth light emitters 173, 174 may be perfectly aligned and the seventh and eighth light emitters 177, 178 may be perfectly aligned, but the seventh and eighth light emitters 177, 178 may be located further from the longitudinal axis A-A than the third and fourth light emitters 173, 174. Finally, a third axis E-E that is parallel to the longitudinal axis A-A may intersect the fifth, sixth, and ninth light emitters 175, 176, 179. The fifth and sixth light emitters 175, 176 may be aligned, and the ninth light emitter 179 may be offset/staggered relative to the fifth and sixth light emitters 175, 176 in the direction of the top edge 168 of the flexible sheet 161.

Stated another way, the first and second light emitters 171, 172 are located a first distance D1 from the longitudinal axis A-A, the third and fourth light emitters 173, 174 are located a second distance D2 from the longitudinal axis A-A, the fifth and sixth light emitters 175, 176 are located a third distance D3 from the longitudinal axis A-A, the seventh and eighth light emitters 177, 178 are located a fourth distance D4 from the longitudinal axis A-A, and the ninth light emitter 179 is located a fifth distance D5 from the longitudinal axis A-A. The fifth distance D5 may be greater than the third distance D3, the third distance D3 may be greater than the fourth distance D4, the fourth distance D4 may be greater than the second distance D2, and the second distance D2 may be greater than the first distance D1.

Thus, the distance between the longitudinal axis A-A and the light emitters 164 of the first row 170 does not continually increase moving from the first side edge 166 or the second side edge 167 towards the vertical centerline B-B. Rather, the distance measured from the longitudinal axis A-A to the light emitters 164 of the first row 170 increases between at least one adjacent pair of light emitters 164 of the first row 170 and decreases between at least one adjacent pair of light emitters 164 of the first row 170 moving in a direction from the first side edge 166 of the flexible sheet 161 towards the vertical centerline B-B of the flexible sheet 161 and moving in a direction from the second side edge 167 of the flexible sheet 161 towards the vertical centerline B-B of the flexible sheet 161. For example, the distance from the longitudinal axis A-A increases from the first light emitter 171 to the third light emitter 173 that is adjacent to the first light emitter 171 moving in a direction from the first side edge 161 towards the vertical centerline B-B, but decreases from the fifth light emitter 175 to the seventh light emitter 177 that is adjacent to the fifth light emitter 175 moving in the direction from the first side edge 166 towards the vertical centerline B-B.

The light emitters 164 of the second row 180 may be arranged in exactly the same relative manner as the light emitters of the first row 170. That is, each of the light emitters 164 of the second row 180 may be staggered in a direction between the top and bottom edges 168, 169 of the flexible sheet 161 relative to each adjacent one of the light emitters 164 of the second row 180. Furthermore, the distance from the longitudinal axis A-A to the light emitters 164 of the second row 180 does not continuously increase moving from the first side edge 166 to the vertical centerline B-B or from the second side edge 167 to the vertical centerline B-B. Rather, the distance from the longitudinal axis A-A to the light emitters 164 of the second row 180 increases between at least one pair of the light emitters 164 and decreases between at least one pair of the light emitters 164 moving in either direction towards the vertical centerline B-B.

Moving in a direction from the first side edge 166 and the second side edge 167 inwardly towards the vertical axis B-B, the second row 180 of light emitters comprises a tenth light emitter 181 adjacent to the first side edge 166 of the flexible sheet 161 and an eleventh light emitter 182 adjacent to the second side edge 167 of the flexible sheet 161. The tenth and eleventh light emitters 181, 182 may be located a sixth distance D6 from the longitudinal axis A-A. The second row 180 of light emitters comprises a twelfth light emitter 183 adjacent to the tenth light emitter 181 and a thirteenth light emitter 184 adjacent to the eleventh light emitter 182. The twelfth and thirteenth light emitters 183, 184 may be located a seventh distance D7 from the longitudinal axis A-A. The second row 180 of light emitters comprises a fourteenth light emitter 185 adjacent to the twelfth light emitter 183 and a fifteenth light emitter 186 adjacent to the thirteenth light emitter 184. The fourteenth and fifteenth light emitters 185, 186 may be located an eighth distance D8 from the longitudinal axis A-A. The second row 180 of light emitters comprises a sixteenth light emitter 187 adjacent to the fourteenth light emitter 185 and a seventeenth light emitter 188 adjacent to the fifteenth light emitter 186. The sixteenth and seventeenth light emitters 187, 188 may be located a ninth distance D9 from the longitudinal axis A-A. The second row 180 of light emitters comprises an eighteenth light emitter 189 between the sixteenth and seventeenth light emitters 187, 188. The eighteenth light emitter 189 may be located a tenth distance D10 from the longitudinal axis A-A. The tenth distance D10 may be greater than the eighth distance D8, the eighth distance D8 may be greater than the ninth distance D9, the ninth distance D9 may be greater than the seventh distance D7, and the seventh distance D7 may be greater than the sixth distance D6.

While there are eighteen light emitters 164 in the exemplified embodiment, the invention is not to be so limited in all embodiments and fewer or a greater number of light emitters 164 may be used in other embodiments. Moreover, if a different number of light emitters is used, this may result in a modification to the specific positioning of the light emitters along the flexible sheet 161 and relative to one another. Thus, variations in the number and positioning of the light emitters may be possible in some embodiments.

The positioning of the light emitters 164 of the first and second rows 170, 180 in this staggered arrangement may optimize coverage of the teeth with the light emitted by the light emitters 164 during a tooth whitening treatment procedure. Of course, slight variations from the specific differences in distance and staggering of the various light emitters 164 may fall within the scope of the invention claimed herein. The invention is not to be limited by the specific distances, spacings, and the like between the light emitters 164 and each other and between the light emitters 164 and the longitudinal axis A-A in all embodiments.

The light emitters 164 may be configured to emit light at a wavelength between 390 nm and 430 nm, more specifically between 400 nm and 420 nm, and still more specifically approximately 410 nm (approximately in this context meaning plus or minus 5 nm). In some embodiments, the light emitted from the light emitters 164 may have a wavelength of exactly 410 nm.

Referring to FIGS. 5, 9 and 10, the lens member 200 will be further described. As noted above, the lens member 200 is formed from a very flexible thermoplastic elastomer, such as one with a Shore A hardness in a range of 30 to 40. Moreover, the lens member 200 may be transparent so that light emitted from the light emitters 164 passes through the lens member 200 to treat the teeth.

The lens member 200 comprises the front surface 201, the rear surface 202, a first side edge 203, a second side edge 204, a top edge 205, and a bottom edge 206. The lens member 200 comprises a vertical centerline Z-Z that extends between the top and bottom edges 204, 205 at a location that is equidistant to the first and second side edges 201, 202. The lens member 200 comprises the first bite protrusion 210 located on a first side of the vertical centerline Z-Z and the second bite protrusion 230 located on a second side of the vertical centerline Z-Z. Each of the first and second bite protrusions 210, 230 protrudes or extends from the front surface 201 and is configured to be held between the user’s top/bottom teeth. The first bite protrusion 210 protrudes from the front surface 201 adjacent to the first side edge 203 and the second bite protrusion 230 protrudes from the front surface 201 adjacent to the second side edge 204. The first and second bite protrusions 210, 230 are spaced apart from one another along the width of the lens member 200 in a direction between the first and second side edges 203, 204. In fact, the gap between the first and second bite protrusions 210, 230 may be larger than the width of the first and second bite protrusions 210, 230.

That is, the first bite protrusion 210 has a first maximum width W1 measured in a direction between the first and second side edges 203, 204 of the lens member 200 and the second bite protrusion 210 has a second maximum width W2 measured in the direction between the first and second side edges 203, 204 of the lens member 200. The first and second maximum widths W1, W2 may be the same, although this is not required in all embodiments. Furthermore, an exposed portion of the front surface 201 of the lens member 200 is located between the first and second bite protrusions 210, 230, with the exposed portion of the front surface 201 being free or devoid of any protrusions thereon. That is, there are no bite protrusions protruding from the front surface 201 in the region between the first and second bite protrusions 210, 230. Thus, the first and second bite protrusions 210, 230 are spaced apart by a distance W3, the distance W3 being a third width of the exposed portion of the front surface 201 of the lens member 200 located between the first and second bite protrusions 210, 230. The distance W3 is greater than each of the first and second maximum widths W1, W2. More specifically, the distance W3 may be greater than a sum of the first and second widths W1, W2. Thus, more than half of the width of the front surface 201 of the lens member 200 does not have any bite protuberance thereon.

In some embodiments, the exposed portion of the front surface 201 which extends between the first and second bite protrusions 210, 230 may have a radius of curvature between 35 mm and 40 mm.

The first bite protrusion 210 comprises a distal edge 211, a first side edge 212 that extends from the front surface 201 to the distal edge 211, and a second side edge 213 that extends from the front surface 201 to the distal edge 211. The first side edge 212 is the edge of the first bite protrusion 210 that is closest to the vertical centerline Z-Z of the lens member 200. The first side edge 212 may be linear. The second side edge 213 of the first bite protrusion 210 may be rounded and may extend to/connect with the first side edge 203. The distal edge 211 of the first bite protrusion 210 may be linear. The first side edge 212 of the first bite protrusion 210 may be oriented at an obtuse angle relative to the distal edge 211 as it extends from the distal edge 211 towards the front surface 201 of the lens member 200. Alternatively, the first side edge 212 may be perpendicular to the distal edge 211.

The second bite protrusion 230 comprises a distal edge 231, a first side edge 232 that extends from the front surface 201 to the distal edge 231, and a second side edge 233 that extends from the front surface 201 to the distal edge 231. The first side edge 232 is the edge of the second bite protrusion 230 that is closest to the vertical centerline Z-Z of the lens member 200. The first side edge 232 may be linear. The second side edge 233 of the second bite protrusion 230 may be rounded and may extend to/connect with the second side edge 204. The distal edge 231 of the second bite protrusion 230 may be linear. The first side edge 232 of the second bite protrusion 230 may be oriented at an obtuse angle relative to the distal edge 231 as it extends from the distal edge 231 towards the front surface 201 of the lens member 200. Alternatively, the first side edge 232 may be perpendicular to the distal edge 231.

The first side edge 212 of the first bite protrusion 210 and the second side edge 232 of the second bite protrusion 230 may diverge from one another with increasing distance from the front surface 201 of the lens member 200 towards the distal ends 211, 231 of the first and second bite protrusions 230. Furthermore, the distal ends 211, 231 of the first and second bite protrusions 210, 230 may be linear and lie in a common plane. In the exemplified embodiment, the first and second bite protrusions 210, 230 have a constant thickness. In other embodiments, the thickness of the first and second bite protrusions 210, 230 may taper moving from the distal ends 211, 231 towards the front surface 201 of the lens member 200. In the exemplified embodiment, the top and bottom surfaces of the first and second bite protrusions 210, 230 are flat/planar. In other embodiments, the top and/or bottom surfaces of the first and second bite protrusions 210, 230 may be linear but angled, wavy, undulated, or comprise some sort of texture, bumps, or the like to increase friction with the teeth and the ability of the user to grip the bite protrusions 210, 230 between the teeth during use.

The lens member 200 may comprise a side edge axis W-W. The side edge axis W-W may intersect the second side edge 213 of the first bite protrusion 210 and the second side edge 233 of the second bite protrusion 230. The side edge axis W-W may intersect the first side edge 212 of the first bite protrusion 210 and the first side edge 232 of the second bite protrusion 230. The side edge axis W-W- may intersect the distance W3 located between and spacing apart the first and second bite protrusions 210, 230. The second side edge 213 of the first bite protrusion 210 and the second side edge 233 of the second bite protrusion 230 may be offset from each other by a side edge distance DX.

The second side edge 213 of the first bite protrusion 210 and the second side edge 233 of the second bite protrusion 230 may be separated by a first side edge distance DX when a first external force is applied to the second side edge 213 of the first bite protrusion 210 and the second side edge 233 of the second bite protrusion 230 along the side edge axis W-W - whereby the first external force may be 0.0 N (zero force).

The second side edge 213 of the first bite protrusion 210 and the second side edge 233 of the second bite protrusion 230 may be separated by a second side edge distance DX when a second external force is applied to the second side edge 213 of the first bite protrusion 210 and the second side edge 233 of the second bite protrusion 230 along the side edge axis W-W.

In some embodiments, the second side edge distance may be about 5 mm less than the first side edge distance when the second external force is less than about 1.5 N. More specifically, in some embodiments, the second side edge distance may be about 5 mm less than the first side edge distance when the second external force is about 0.97 N. In some embodiments, the second side edge distance may be about 10 mm less than the first side edge distance when the second external force is less than about 2.5 N. More specifically, in some embodiments, the second side edge distance may be about 10 mm less than the first side edge distance when the second external force is about 1.35 N. In some embodiments, the second side edge distance may be about 15 mm less than the first side edge distance when the second external force is less than about 3.5 N. More specifically, in some embodiments, the second side edge distance may be about 15 mm less than the first side edge distance when the second external force is about 1.58 N. In some embodiments, the second side edge distance may be about 20 mm less than the first side edge distance when the second external force is less than about 4.5 N. More specifically, in some embodiments, the second side edge distance may be about 20 mm less than the first side edge distance when the second external force is about 1.84 N. In some embodiments, the second side edge distance may be about 25 mm less than the first side edge distance when the second external force is less than about 5.5 N. More specifically, in some embodiments, the second side edge distance may be about 25 mm less than the first side edge distance when the second external force is about 2.54 N. As used herein, the term about may equate to the numerical value provided plus or minus 5% of that value.

The bite protrusions 210, 230 may be omitted in some embodiments. However, it has been found that including such bite protrusions 210, 230 has advantages in the tooth whitening process for many users. In particular, when the bite protrusions 210, 230 are omitted, the user may have the top teeth overlapping the bottom teeth during use of the oral cavity treatment device 1000, which may create a shadow region along the bottom teeth that are covered by the top teeth. These bottom teeth may then not receive the light from the light emitters as effectively as the other teeth which may cause these bottom teeth that are covered/shadowed to obtain an inferior whitening result relative to the other teeth. The bite protrusion 210, 230 may encourage a user to keep the top and bottom teeth slightly apart so that the top teeth do not cover the bottom teeth (or vice versa, depending on a particular person’s bite) and there is no shadow. This ensure consistent coverage of all of the teeth with the light emitted by the light emitters 164.

Moreover, by having the discrete bite protrusions 210, 230 rather than a singular bite plate extending continuously from one end of the lens member 200 to the other, additional advantages are achieved. In particular, users have indicated that with a singular bite plate, they are more likely to salivate during use of the device, which can lead to discomfort. The bite protrusions 210, 230 described and illustrated herein may reduce user salivation during use of the device.

In the exemplified embodiment, the lens member 200 has a height measured between the top and bottom ends 205, 206 at the vertical centerline Z-Z which is intentionally selected for user comfort. The height may fall in a range of 20 mm to 25 mm, more specifically 21 mm to 24 mm, and still more specifically 22 mm to 23 mm. For some user’s, depending on mouth size, the user’s top and bottom (superior and inferior) labial frenulum may bottom out on the top and bottom ends 205, 206 of the lens member 200 without the user’s teeth engaging the bite protrusions 210, 230. In any case, the height of the lens member 200 has been specifically selected to maximize comfort while ensuring adequate and complete coverage of all teeth desired for whitening.

The rear surface 202 of the lens member 200 comprises a recess region 240 and an annular lip 241 that surrounds the recess region 240. The lens member 200 comprises a plurality of pockets 242 formed into the rear surface 202 within the recess region 240. Each of the pockets 242 is configured to receive one of the light emitters 164 of the lamp 160 when the oral cavity treatment device 1000 is assembled. FIG. 6 perhaps best illustrates some of the light emitters 164 nesting within the pockets 242. The lens member 200 also comprises the blind holes 245 in the rear surface 202 along the recess region 240. As noted above, the blind holes 245 are configured to receive the protrusions 255 of the strain relief member 250 when the oral cavity treatment device 1000 is assembled, as best shown in FIG. 6. None of the pockets 242 nor the blind holes 245 extends through the full thickness of the lens member 200. That is, none of the pockets 242 nor the blind holes 245 extend to the front surface 201 of the lens member 200.

As best seen in FIG. 5, the lens member 200 and the support panel 211 of the support member 210 have engaging surfaces that cooperate to help maintain the bond between those two components. In the exemplified embodiment, the lens member 200 comprises a V-shaped protrusion 248 that nests within a V-shaped notch 249 in the peripheral edge of the support panel 211. In other embodiments, the lens member 200 may comprise the notch and the support panel 211 may comprise the protrusion. Furthermore, the shape of the notch 249 and protrusion 248 could be modified from that which is shown in the drawings while still achieving an interlocking engagement between the components.

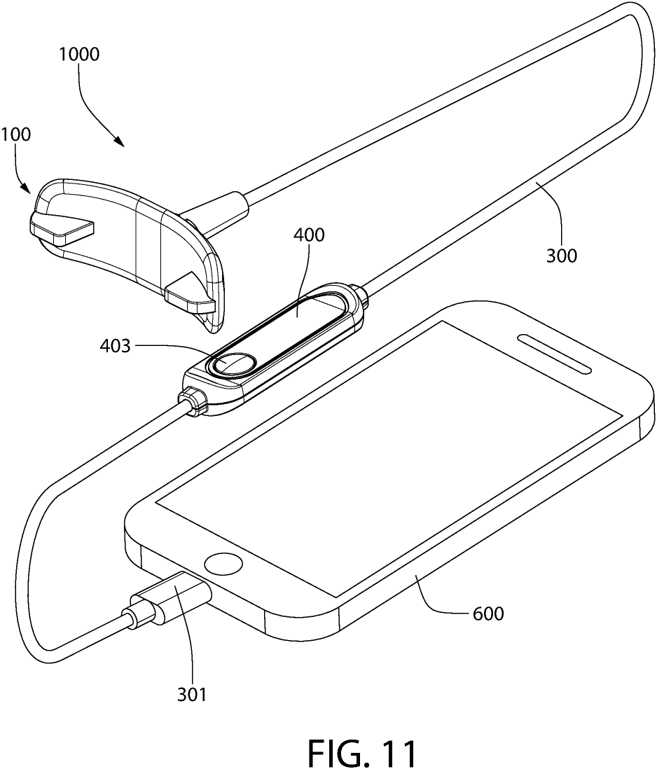

Referring to FIG. 11, the oral cavity treatment device 1000 is illustrated coupled to an electronic device 600. Specifically, the plug 301 of the power cord 300 is plugged into a socket of the electronic device 600 so that power may be supplied from the electronic device 600 to the oral cavity treatment device 1000 to power on the light emitters 164 as described herein. With the plug 301 of the power cord 300 plugged into the socket of the electronic device 600, a user can actuate the actuator 403 on the control unit 400 to initiate a treatment session. Actuation of the actuator 403 may result in the light emitters 164 illuminating for a predetermined period of time as determined by the timer, processor, or the like of the control unit 400. In the exemplified embodiment, the electronic device 600 is a smart phone. As discussed above, the invention is not to be so limited in all embodiments and the electronic device 600 may take on other forms such as being any of the types of devices or power supply sources described above.

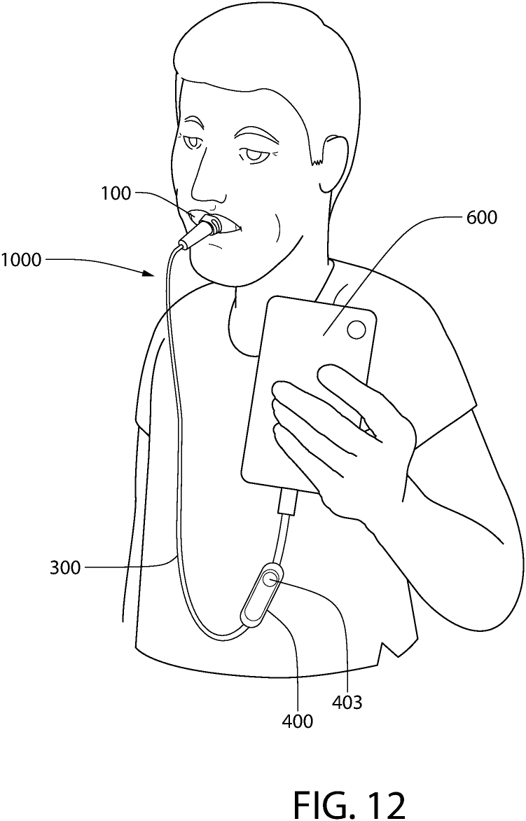

Referring to FIG. 12, the plug 301 of the power cord 300 is plugged into the electronic device 600 and the mouthpiece 100 of the oral cavity treatment device 1000 is positioned within the user’s oral cavity. The user then activates the actuator 403 to start the treatment protocol. Actuating the actuator 403 may cause the light emitters 164 to emit light for a predetermined period of time, as discussed herein. The predetermined period of time may be 5 minutes, 10 minutes, 15 minutes, 20 minutes, 25 minutes, 30 minutes, or any other desired time period. The power cord 300 has a length of between 30 and 40 inches, or between 32 and 36 inches, as described above. This length should be sufficient to enable the user to store their phone in a rear pocket of their pants while the mouthpiece 100 is positioned within their mouth.

Referring to FIGS. 13A and 13B, an exemplary embodiment of a control unit 700 is illustrated. The control unit 700 is identical to the control unit 400 described above, except for the differences specifically described herein. Thus, the control unit 700 comprises a housing 702 having an outer surface 702 and an actuator 703 located along the outer surface 702. The actuator 703 is a depressible button in the exemplified embodiment. The control unit 700 also comprises a processor, a timer, and any other electronic devices required for proper operation and functionality of the device as has been described herein. The processor, timer, and other electronic devices may be located within an interior of the housing 701.

In this embodiment, the outer surface 702 comprises a display 710. Furthermore, in the exemplified embodiment the display 710 comprises a plurality of visual indicators 711a-d that may be illuminated to provide a visual indication of a particular selected mode of operation associated with the use of the oral cavity treatment device 1000. In FIG. 13A, just one of the visual indicators 711a is illuminated, whereas in FIG. 13B all four of the visual indicators 711a-d are illuminated. While there are four visual indicators 711a-d in the exemplified embodiment there could be fewer in other embodiments. The number of visual indicators 711a-d may correspond with and match the number of possible modes that the user selects between.

To switch the device between the modes, the user may activate the actuator 703 for a first period of time. That is, the user will quickly press the actuator 703 to change the mode of operation prior to initiating a treatment protocol. The user can then activate the actuator 703 for a second period of time (a longer period of time) to initiate the treatment protocol associated with the selected mode. In an alternative embodiment the control unit 700 may comprise two actuator. In such an embodiment, actuation of a first one of the actuators may switch between the modes and actuation of a second one of the actuators may power the device on/off to initiate a treatment protocol.

In some embodiments, the different modes are associated with a different treatment time for a treatment protocol. The plurality of modes may comprise a first mode whereby the plurality of light emitters are powered on for a first predetermined period of time, a second mode whereby the plurality of light emitters are powered on for a second predetermined period of time, and a third mode whereby the plurality of light emitters are powered on for a third predetermined period of time, the first, second, and third predetermined periods of time being preset and being different from one another. For example, in the first mode the treatment time may be 5 minutes, in the second mode the treatment time may be 10 minutes, in the third mode the treatment time may be 15 minutes, and in the fourth mode the treatment time may be 30 minutes. Of course, other set times may be associated with each of the modes and the set times noted herein are exemplary. For example, the different times could be 5, 10, 15, and 20 minutes in an alternative embodiment. Thus, the mode selection provides the user with a choice of “lights on” time for a given treatment session, and a user can change the “lights on” time from session to session.

There may be occasions where the user has more time available to conduct a more prolonged whitening treatment with the expectations of a faster whitening result. Thus, the mode selection provides the switch/timer controller with a toggle option to extend the time the light emitters 164 remain on, and therefore extend the impact of their energy on the applied formula, leading to an accelerated whitening outcome. In-vitro tests on stained bovine teeth have demonstrated that the number of treatments necessary to deliver a given visible whitening result for a 5-minute ‘lights-on’ time is 11, whereas this reduces to 8 treatments when the timer extends the ‘lights-on’ to 10 minutes, 5 treatments when extended to 15 minutes and 3 treatments when extended to 30 minutes. Providing this level of user flexibility in effect provides for a customizable whitening experience. The oral cavity treatment device 1000 described herein is comfortable, conformable, and flexible, which enables this customization by ensuring that, unlike many more rigid or heavy devices, even prolonged ‘in-mouth’ usage periods are comfortable enough to ensure compliance and therefore a better chance of delivering against consumers’ whitening expectations.

Furthermore, the feature of personalizing the “lights on” time for a given session may form just one aspect of a user-customization feature. This variable of “lights on” time may be used in combination with a choice of peroxide level in the tooth whitening formula coated onto the teeth prior to the light treatment. In some embodiments, there may be a dedicated software application (“app”) used with the oral cavity treatment device 1000. Using the app, the user may highlight an event date in the future (e.g., a wedding in a week, an interview next Tuesday, a date this upcoming weekend), and the app may recommend a particular formula, light-on time, and power output to achieve the user’s whitening goals within the user’s timeframe. Note that in addition to adjusting light-on time, the control unit 700 may also allow for adjusting the brightness or power for the specific wavelength of light emitted by the light emitters 164. This may assist in maximizing the speed of achieving the whitening outcome.

Thus, the personalized approach may entail three elements of about three or four settings: light-on time (3 or 4 distinct pre-set time periods), LED power setting (3 or 4 different levels), and choice of peroxide level (3 or four different levels or weight percentages). While some people want to achieve a maximum whitening benefit in the shortest amount of time, this is not the goal for all users. Rather, for others the desire is for to achieve maximum whitening while maintaining comfort. Concerns related to whitening discomfort may be alleviated by choosing a lower peroxide level, but longer light-on time, and increased power output, perhaps together with an increased number of treatments.

In an embodiment, the mouthpiece 100 may be configured to be custom molded by the end user. In particular, the mouthpiece 100 of the oral cavity treatment device 1000 may be formed from a material that is capable of being molded when heated and then maintaining its molded shape when cooled from the heated condition. Thus, a user may place the mouthpiece 100 into a hot liquid (e.g., hot water) and then position the mouthpiece 100 into the user’s mouth. When heated and then placed into the user’s mouth, the mouthpiece 100 may be molded and/or fitted to the user’s bite. Once this is complete, the user may remove the mouthpiece 100 from his or her mouth and allow the mouthpiece 100 to cool. Once cooled, the mouthpiece 100 may maintain its molded shape in accordance with the user’s bite and other mouth features.

As noted, in this embodiment the oral cavity treatment device 1000 comprises a power cord 300 that is attached to the mouthpiece 100. This differs from conventional athletic mouthguards that have custom molding capabilities. In some embodiments, it may be desirable to not submerge the power cord 300 into the hot water while the mouthpiece 100 is submerged into the hot water to prevent damaging the electric wires of the power cord 300. In some embodiments, techniques, processes, and/or structures may be available and used to submerge the mouthpiece 100 into hot water for a desired period of time without also submerging the power cord 300. In some embodiments, when submerging the mouthpiece 100 into the hot water care should be taken to avoid submerging the control unit 400 into the hot water because doing so could destroy the electronics of the control unit 400 and render the device inoperable.

The invention set forth herein may be directed to a method of whitening teeth. The method may include the following steps: selecting a tooth whitening composition from a plurality of tooth whitening compositions having different weight percentages of an active ingredient; applying the tooth whitening composition onto teeth of a user; selecting a brightness at which a plurality of light emitters of an oral cavity treatment device will emit light when activated; selecting a duration during which the plurality of light emitters of the oral cavity treatment device will emit the light when activated; positioning a mouthpiece of the oral cavity treatment device into an oral cavity of the user; and activating the oral cavity treatment device so that the plurality of light emitters emit the light onto the teeth of the user that are pre-coated with the tooth whitening composition.

In some embodiments, the user may simply apply a tooth whitening composition onto the teeth without selecting a tooth whitening composition from a plurality of tooth whitening compositions having different weight percentages of the active ingredient. In some embodiments, the user may not be able to adjust the brightness of the light emitters, but may only be able to select the duration at which the light emitters emit the light during a treatment session. Thus, any of the steps identified above may be omitted from the method of whitening teeth in various different embodiments of the invention described herein.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

### EXAMPLES

A series of tests were performed on a number of oral cavity treatment devices - including an oral cavity treatment device of the present invention - to test the mechanical strength and related flexibility and resulting comfort on the user.

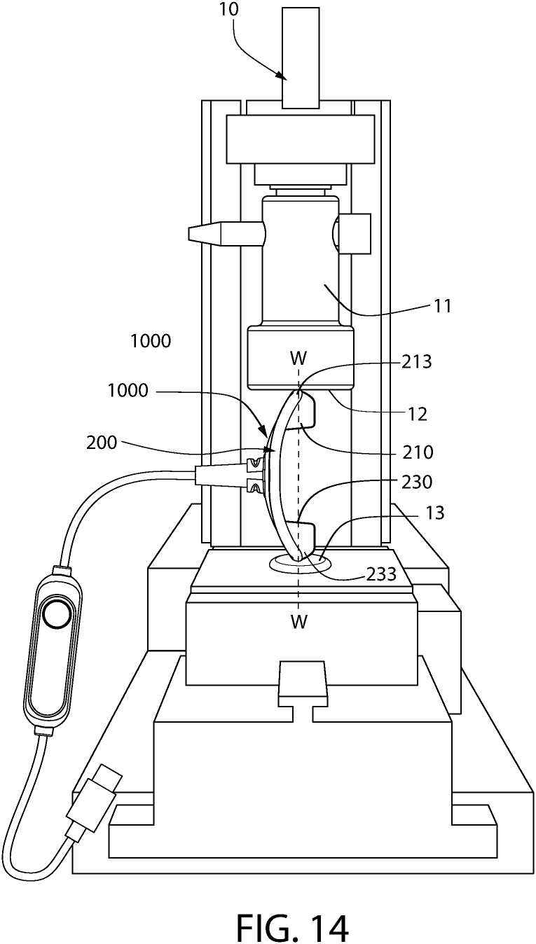

Referring to FIG. 14, the testing arrangement of the oral cavity treatment device 1000 comprising a lens 200 as well as comparative oral cavity treatment devices will be discussed in further detail. Each oral cavity treatment device was positioned in a compression testing machine 10 that comprises a load cell 11 having an upper sample holder 12 and a lower sample holder 13. During testing, each oral cavity treatment device is positioned between the upper and lower sample holders 12, 13 such that the second side edge 213 of the first bit protrusion 210 contacts the upper sample holder 12 and the second side edge 233 of the second bit protrusion 230 contacts the lower sample holder 13.

Once properly positioned, the load cell 11 applies a controlled load to the oral cavity treatment device such that the upper sample holder 12 moves closer to the lower sample holder 13 thereby compressing the oral cavity treatment device 1000 along the side edge axis W-W of the lens 200.

Each oral cavity treatment device 1000 is subjected to a controlled load displacement cycle (i.e., deformation) of 5 mm, 10 mm, 15 mm, 20 mm, and 25 mm. The amount of force (measured in Newtons) required for each displacement cycle is then recorded in, as set forth below in Table 1 - as referred to as “load distance” or “distance under load”.

TABLE 1

| | | | | | |
|---|---|---|---|---|---|
| | Cycle 1 (5 mm) | Cycle 2 (10 mm) | Cycle 3 (15 mm) | Cycle 4 (20 mm) | Cycle 5 (25 mm) |
| Ex. 1 | 0.97 N | 1.35 N | 1.58 N | 1.84 N | 2.54 N |
| Comp. Ex. 1 | 2.09 N | 3.65 N | 5.73 N | 7.68 N | 9.88 N |
| Comp. Ex. 2 | 1.78 N | 2.76 N | 3.84 N | 4.86 N | 5.94 N |
| Comp. Ex. 3 | 1.8 N | 3.2 N | 4.79 N | 6.11 N | 7.1 N |
| Comp. Ex. 4 | 2.27 N | 4.14 N | 6.53 N | 8.6 N | 10.2 N |
| Comp. Ex. 5 | 2.16 N | 3.98 N | 6.23 N | 8.35 N | 10.11 N |
| Comp. Ex. 6 | 13.62 N | 29.3 N | 58.26N | 90.15 N | 197 N |

As demonstrated by Table 1, the oral cavity treatment device 1000 of the present invention (i.e., Ex. 1) is capable of undergoing a displacement cycle with a substantially lower amount of force required of Comp. Ex. 1-6. Specifically, the force required for the displacement of Ex. 1 is an order of magnitude less than each of Comparative Examples 4-6. The lower force required to achieve the same displacement cycle of Ex. 1 reflects the high degree of flexibility of the mouth piece of the oral cavity treatment device 1000 of the present invention, as compared to relatively more rigid oral care treatment devices of Comp. Ex. 1-6.

Further, the oral cavity treatment device of Ex. 1 and Comp. Ex. 6 were then tested for comfort by users with a large mouth size - whereby a passing grade was assigned to mouth comfort that does not result in any gum pain or excess effort to hold inside of the mouth cavity for a treatment period of time. The results of the comparison are set forth below in Table 2.

TABLE 2

| | | |
|---|---|---|
| | Ex. 1 | Comp. Ex. 6 |
| Mouth Comfort | Pass | Fail |

As demonstrated by Table 2, the oral cavity treatment device of the present invention resulted in a passing grade while the oral cavity treatment device of Comp. Ex. 6 resulted in a failing grade - further signifying that the high degree of flexibility of the oral cavity treatment device of the present invention results in an unexpected improvement over previous oral cavity treatment devices that are comparatively more rigid.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

What is claimed is:

1. An oral cavity treatment device comprising:
a mouthpiece comprising:
a lamp comprising a flexible sheet having a first side edge, a second side edge, and a top edge and a bottom edge each extending between the first and second side edges and a plurality of light emitters coupled to the flexible sheet, the plurality of light emitters comprising a first row of light emitters located adjacent to the top edge of the flexible sheet and arranged in a spaced apart manner in a direction between the first and second side edges of the flexible sheet, the first row of light emitters comprising a first light emitter located adjacent to the first side edge of the flexible sheet, a second light emitter located adjacent to the second side edge of the flexible sheet, and a plurality of central light emitters located between the first and second light emitters, the first and second light emitters being intersected by a first axis that extends between the first and second side edges of the flexible sheet, and wherein each of the central light emitters is located between the first axis and the top edge of the flexible sheet without being intersected by the first axis; and
a lens member covering the front surface of the flexible sheet.

2. The oral cavity treatment device according to claim 1 wherein none of the plurality of light emitters is located between the first light emitter and the first side edge of the flexible sheet or between the second light emitter and the second side edge of the flexible sheet.

3. The oral cavity treatment device according to claim 1 further comprising a second axis that is parallel to the first axis and located between the first axis and the top edge of the flexible sheet, the second axis intersecting a third light emitter that is adjacent to the first light emitter and a fourth light emitter that is adjacent to the second light emitter, and wherein at least one of the plurality of central light emitters that is located between the third and fourth light emitters is positioned between the second axis and the top edge of the flexible sheet.

4. The oral cavity treatment device according to claim 3 further comprising a fifth light emitter adjacent to the third light emitter, a sixth light emitter adjacent to the fourth light emitter, a seventh light emitter adjacent to the fifth light emitter, and an eighth light emitter adjacent to the sixth light emitter, wherein the second axis intersects the third, fourth, seventh, and eighth light emitters, and wherein the fifth and sixth light emitters are located between the second axis and the top edge of the flexible sheet.

5. The oral cavity treatment device according to claim 4 wherein the third and fourth light emitters are aligned in a direction between the top and bottom edges of the flexible sheet and wherein the seventh and eighth light emitters are aligned in the direction between the top and bottom edges of the flexible sheet, the seventh and eighth light emitters being staggered in the direction between the top and bottom edges of the flexible sheet relative to the third and fourth light emitters.

6. The oral cavity treatment device according to claim 4 further comprising a ninth light emitter located between the seventh and eighth light emitters, and wherein a third axis that is parallel to the first and second axes and located between the second axis and the top end of the flexible sheet intersects the fifth, sixth, and ninth light emitters, wherein the fifth and sixth light emitters are aligned in the direction between the top and bottom edges of the flexible sheet and wherein the ninth light emitter is staggered in the direction between the top and bottom edges of the flexible sheet relative to the fifth and sixth light emitters.

7. (canceled)

8. The oral cavity treatment device according to claim 1 wherein the flexible sheet has a longitudinal axis extending between the first and second side edges and located equidistantly between the top and bottom edges, wherein the first and second light emitters are spaced a first distance from the longitudinal axis, and wherein each of the plurality of central light emitters is located a distance from the longitudinal axis that is greater than the first distance.

9. The oral cavity treatment device according to claim 8 further comprising:
a third light emitter adjacent to the first light emitter, a fourth light emitter adjacent to the second light emitter, a fifth light emitter adjacent to the third light emitter, and a sixth light emitter adjacent to the fourth light emitter, the third and fourth light emitters being spaced a second distance from the longitudinal axis and the fifth and sixth light emitters being spaced a third distance from the longitudinal axis, the third distance being greater than the second distance and the second distance being greater than the first distance;
a seventh light emitter adjacent to the fifth light emitter and an eighth light emitter adjacent to the sixth light emitter, the seventh and eighth light emitters located a fourth distance from the longitudinal axis, the fourth distance being greater than the second distance and less than the third distance; and
a ninth light emitter located between the seventh and eighth light emitters, the ninth light emitter being located a fifth distance from the longitudinal axis, the fifth distance being greater than the third distance.

10. (canceled)

11. (canceled)

12. The oral cavity treatment device according to claim 1 wherein the plurality of light emitters comprises a second row of light emitters arranged in a spaced apart manner in a direction between the first and second side edges of the flexible sheet, the first row of light emitters being adjacent to the top end of the flexible sheet and the second row of light emitters being adjacent to the bottom end of the flexible sheet, wherein the flexible sheet has a longitudinal axis extending between the first and second side edges and located equidistantly between the top and bottom edges, and wherein the second row of light emitters comprises:
a tenth light emitter adjacent to the first side edge of the flexible sheet and an eleventh light emitter adjacent to the second side edge of the flexible sheet, the tenth and eleventh light emitters being located a sixth distance from the longitudinal axis;
a twelfth light emitter adjacent to the tenth light emitter and a thirteenth light emitter adjacent to the eleventh light emitter, the twelfth and thirteenth light emitters being located a seventh distance from the longitudinal axis;
a fourteenth light emitter adjacent to the twelfth light emitter and a fifteenth light emitter adjacent to the thirteenth light emitter, the fourteenth and fifteenth light emitters being located an eighth distance from the longitudinal axis;
a sixteenth light emitter adjacent to the fourteenth light emitter and a seventeenth light emitter adjacent to the fifteenth light emitter, the sixteenth and seventeenth light emitters being located a ninth distance from the longitudinal axis;
an eighteenth light emitter between the sixteenth and seventeenth light emitters, the eighteenth light emitter being located a tenth distance from the longitudinal axis; and
wherein the tenth distance is greater than the eighth distance, the eighth distance is greater than the ninth distance, the ninth distance is greater than the seventh distance, and the seventh distance is greater than the sixth distance.

13-28. (canceled)

29. An oral cavity treatment device comprising:
a mouthpiece comprising:
a lamp comprising a flexible sheet having a front surface and plurality of light emitters located on the front surface of the flexible sheet; and
a lens member covering the front surface of the flexible sheet and being transparent so that light emitted by the plurality of light emitters passes through the lens member, the lens member comprising a front surface having a vertical centerline, a first bite protrusion extending from the front surface of the lens member on a first side of the vertical centerline and having a first maximum width, and a second bite protrusion extending from the front surface of the lens member on a second side of the vertical centerline and having a second maximum width, an exposed portion of the front surface of the lens member located between the first and second bite protrusions being devoid of any additional bite protrusions and having a third width that is greater than each of the first and second maximum widths.

30. The oral cavity treatment device according to claim 29 wherein the third width is greater than a sum of the first and second widths.

31. (canceled)

32. The oral cavity treatment device according to claim 29 wherein at least a portion of the exposed portion of the front surface of the lens member has a radius of curvature of between 35 mm and 40 mm.

33. The oral cavity treatment device according to claim 29 wherein the first bite protrusion comprises a distal edge and a first side edge that is closest to the vertical centerline and extends from the front surface of the lens member to the distal edge of the first bite protrusion, wherein the second bite protrusion comprises a distal edge and a first side edge that is closest to the vertical centerline and extends from the front surface of the lens member to the distal edge of the second bite protrusion, the first side edges of the first and second bite protrusions being linear.

34. The oral cavity treatment device according to claim 33 wherein the first side edges of the first and second bite protrusions diverge from one another with increasing distance from the front surface of the lens member to the distal edges of the first and second bite platforms.

35. The oral cavity treatment device according to claim 33 wherein the distal edges of the first and second bite protrusions are linear and lie in a common plane.

36. (canceled)

37. (canceled)

38. The oral cavity treatment device according to claim 29 wherein the lens member comprises a rear surface opposite the front surface, the rear surface comprising a plurality of pockets that do not extend through to the front surface of the lens member, each of the plurality of light emitters nesting within one of the pockets, and further comprising an annular lip protruding from the rear surface of the lens member so that a recessed region of the lens member in which the plurality of pockets are located is recessed relative to an outer surface of the annular lip, the lamp being disposed within the recessed region of the lens member.

39. (canceled)

40. The oral cavity treatment device according to claim 29 wherein each of the first and second bite protrusions comprises a flat top surface, a flat bottom surface, and a linear distal edge.

41-55. (canceled)

56. An oral cavity treatment device comprising:
a mouthpiece comprising:
a lamp comprising a flexible sheet having a front surface and a rear surface and a plurality of light emitters protruding from the front surface of the flexible sheet;
a support member covering the rear surface of the flexible sheet, the support member having an exposed rear surface; and
a lens member covering the front surface of the flexible sheet, the lens member having an exposed front surface; and
a power cord extending from the mouthpiece, the power cord comprising a plug for engaging a socket of an electronic device to power the plurality of light emitters;
wherein the mouthpiece comprises a first side portion, a second side portion, and a central portion between the first and second side portions; and
wherein the mouthpiece is flexible such that each of the first and second side portions is configured to be folded relative to the central portion in a first direction so that the exposed front surface of the lens member along the first and second side portions contacts the exposed front surface of the lens member along the central portion and a second direction so that the exposed rear surface of the support member along the first and second side portions contacts the exposed rear surface of the support along the central portion.

57. The oral cavity treatment device according to claim 56 further comprising a control unit coupled to the power cord, the control unit comprising an actuator and a processor for powering the plurality of light emitters in accordance with a treatment protocol, the control unit being located closer to the plug of the power cord than to the mouthpiece.

58. The oral cavity treatment device according to claim 56 wherein the support member and the lens member are each formed of a thermoplastic elastomer having a Shore A hardness between 30 and 40.

59-80. (canceled)
